(19) United States
(12) Patent Application Publication
(10) Pub. No.: US 2026/0364226 A1
(43) Pub. Date: Nov. 19, 2026

(54) FORM-FITTING AND BIOCOMPATIBLE HYDROGEL WITH TUNABLE MECHANICAL PROPERTIES

(71) Applicant: Nano and Advanced Materials Institute Limited, Hong Kong (HK)

(72) Inventors: Shengchang TANG, Hong Kong (HK); Lok Hei LI, Hong Kong (HK); Aaron LEUNG, Hong Kong (HK); Wenjun MENG, Hong Kong (HK)

(21) Appl. No.: 19/145,901

(22) PCT Filed: Jan. 3, 2024
(86) PCT No.: PCT/CN2024/070389

§ 371 Date: Jul. 4, 2025

Related U.S. Application Data
(60) Provisional application No. 63/478,504, filed on Jan. 5, 2023.

(51) Int. Cl. A61L 26/00; A61K 8/02; A61K 8/04; A61K 8/73; A61K 8/9794

(52) U.S. Cl. CPC A61L 26/008; A61K 8/0212; A61K 8/042; A61K 8/735; A61K 8/9794; A61L 26/0023; A61L 26/0095

(57) ABSTRACT

A method of manufacturing a form-fitting and biocompatible hydrogel with tunable mechanical properties; particularly, the hydrogel includes a chemical network and a physical network. A monomer, a crosslinker and a photo-initiator or a crosslink-able polymer and a photo-initiator are mixed well to obtain a chemical network precursor, and sodium alginate, calcium carbonate and D-glucono-δ-lactone are mixed to form a physical network precursor with ionic crosslinking. After the preparation of two network precursors, the physical network precursor is introduced into the chemical network precursor to form a moldable double network. With providing a UV light, the chemical network precursor starts to crosslink to form a crosslinked polymer chemical network interengaged with the ionic crosslinked physical network to obtain a form-fitting and biocompatible hydrogel.

### CROSS-REFERENCE TO RELATED APPLICATIONS

The present application claims priority from U.S. provisional patent application Ser. No. 63/478,504 filed Jan. 5, 2023, and the disclosure of which is incorporated herein by reference in its entirety.

### FIELD OF THE INVENTION

The present invention generally relates to polymer materials. More specifically the present invention relates to form-fitting hydrogels with tunable mechanical properties, highly-biocompatible and storage stability.

### BACKGROUND OF THE INVENTION

Hydrogels are primarily applied to various medical products (e.g., wound dressings and drug delivery vehicles), personal care products (e.g., facial masks, contact lens) and other areas. The market size of hydrogels is still growing, and it is expected to reach 15.33 million USD in 2022. There are many commercially available hydrogel products in the market; however, many of them have limited mechanical properties. For example, some soft hydrogels, such as contact lenses, aim to deliver comfort but break easily, because they lack stretchability. On the other hand, in order to maintain a large stretchability to produce facial masks, hydrogels are nearly elastic, resulting in not being sufficiently form-fitting.

Although there are some engineering solutions, such as lamination with non-woven support layers and increasing the polymer concentration, the hydrogels remain far from satisfactory and still have many drawbacks, including reduced water content and load of active ingredients, high product cost, labor-intensive and complex manufacturing, and not being ecofriendly.

There is a need in the art to produce a hydrogel that can possess both great form-fitting properties and satisfactory mechanical properties on a mass scale by a time-saving manufacturing process. At the same time, the hydrogel should possess high biocompatibility and be stable during storage. Therefore, the present invention addresses this need.

### SUMMARY OF THE INVENTION

It is an objective of the present invention to provide a form-fitting and biocompatible hydrogel with tunable mechanical properties and a manufacturing method thereof. Unlike conventional hydrogels that are formed solely via chemical crosslinking, the present hydrogel is composed of both chemically and physically crosslinked networks, where the reversibly “breakable” physical network allows effective energy dissipation, rendering the hybrid hydrogels stretchable, much less fragile, and to change the dimension upon stretching and unloading. Further, the present hydrogel is non-toxic, non-irritating and medically suitable.

In accordance with a first aspect of the present invention, a method of manufacturing a form-fitting and biocompatible hydrogel with tunable mechanical properties is provided. Particularly, the hydrogel includes a chemical network and a physical network. In this method, the gelation process of both chemical and physical networks are well controlled and the detailed process conditions are suitable for mass production. Furthermore, the whole manufacture time is reduced such that fabrication may be completed within 15 minutes.

A monomer, a crosslinker and a photo-initiator or a crosslink-able polymer and a photo-initiator are mixed well to obtain a chemical network precursor, and sodium alginate, calcium carbonate and D-glucono-δ-lactone are mixed to form a physical network precursor with ionic crosslinking. After the preparation of two network precursors, the physical network precursor is introduced into the chemical network precursor to form a moldable double network. With providing a UV light, the chemical network precursor starts to crosslink to form a crosslinked polymer chemical network interengaged with the ionic crosslinked physical network to obtain a form-fitting and biocompatible hydrogel.

In accordance with one embodiment of the present invention, calcium carbonate and D-glucono-δ-lactone are used for controlling the timing of Ca²⁺ availability to achieve homogenous mixing before gelation.

In accordance with another embodiment of the present invention, crosslinking time is shortened to less than 10 minutes by adding the photo-initiator.

In accordance with one embodiment of the present invention, the tunable mechanical properties comprise elastic modulus, elongation at break and residual strain.

In accordance with one embodiment of the present invention, the monomer is acrylamide, the crosslinker is N, N′-methylenebisacrylamide and the crosslink-able polymer is polyethylene glycol diacrylate or diacrylamide.

In accordance with another embodiment of the present invention, the crosslink-able polymer has a molecular weight of 8-20 kDa and a concentration of 5-15% w/v.

In accordance with one embodiment of the present invention, the hydrogel is capable of incorporating hyaluronic acid or aloe vera.

In accordance with one embodiment of the present invention, the concentration of the monomer is 10-20% w/v and the monomer to crosslinker ratio is 100:1000, and the concentration of the photo-initiator is 0.01-0.1% w/v.

In accordance with one embodiment of the present invention, the concentration of sodium alginate is 1-5% w/v, the concentration of calcium carbonate is 7.5-60 mM and the molar concentration of D-glucono-δ-lactone is 15-120 mM, and the stoichiometric ratio of calcium carbonate to D-glucono-δ-lactone is 1:2, wherein the sodium alginate has a viscosity of 50-500 mPa·s at 1%.

In accordance with one embodiment of the present invention, the UV light is selected from UVA, UVB and UVC light; more particularly, the UV light is a UVC light with a wavelength of 200-280 nm.

In accordance with a second aspect of the present invention, a form-fitting and biocompatible hydrogel with tunable mechanical properties is disclosed. Specifically, the hydrogel has a pH value in a range of 4.0-8.5 at room temperature, and the hydrogel is non-cytotoxic and compatible to skin.

In accordance with one embodiment of the present invention, the hydrogel has an elastic modulus in range of 10-50 kPa, an elongation at break in range of 100-300% and a residual strain in range of 0-35%, and the hydrogel is capable of incorporating hyaluronic acid or aloe vera for up to 2 wt %.

In accordance with one embodiment of the present invention, the crosslinked chemical network includes polymers of different molecular weights. It is worth noting that the polymer may be formed during or before the gel-forming crosslinking reaction.

In accordance with a third aspect of the present invention, a wound dressing including the hydrogel disclosed above is provided. Particularly, the wound dressing may be in sterile form.

In accordance with a fourth aspect of the present invention, a facial mask including the hydrogel disclosed above and a skincare solution is provided. For example, the skincare solution includes hyaluronic acid or aloe vera.

### BRIEF DESCRIPTION OF THE DRAWINGS

Embodiments of the invention are described in more details hereinafter with reference to the drawings, in which:

FIG. 1 depicts a flow chart of hydrogel preparation of one embodiment of the present invention; and

FIGS. 2A-2C depict the hydrogel structure at normal state (FIG. 2A), extension state (FIG. 2B) and form-fitting state (FIG. 2C), respectively.

### DETAILED DESCRIPTION

In the following description, a form-fitting and highly-biocompatible hydrogel and the like are set forth as preferred examples. Accordingly, the present invention provides method and applications of a hydrogel comprising a chemical network and physical network. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

In accordance with a first aspect of the present invention, a method of manufacturing a form-fitting and biocompatible hydrogel, including a chemical network and a physical network, with tunable mechanical properties is provided.

In order to manufacture a form-fitting and highly biocompatible hydrogel with tunable mechanical properties, a distinctive approach involves creating a double network structure, combining a chemical network and a physical network. Initially, a chemical network precursor is prepared by mixing a monomer, a crosslinker, and a photo-initiator or a crosslinkable polymer and a photo-initiator. Concurrently, a physical network precursor is formed by mixing sodium alginate, calcium carbonate, and D-glucono-δ-lactone, introducing ionic crosslinking. The physical network precursor is then introduced into the chemical network precursor, creating a moldable double network. UV light is applied to crosslink the chemical network precursor, resulting in a crosslinked polymer chemical network intricately connected with the ionic crosslinked physical network, producing a form-fitting and highly biocompatible hydrogel.

To ensure precise control over the timing of Ca²⁺ availability for homogeneous mixing before gelation, calcium carbonate and D-glucono-δ-lactone are utilized. Specifically, calcium carbonate functions as an ionic crosslinker, while D-glucono-δ-lactone acts as a sequestering agent to slow down the Ca²⁺ crosslinking process. The addition of a photo-initiator serves to expedite crosslinking, reducing the time required to less than 10 minutes. The tunable mechanical properties of the hydrogel, encompassing elastic modulus, elongation at break, and residual strain, are modulated as needed. Key components in this process include the monomer acrylamide, the crosslinker N, N′-methylenebisacrylamide, and the crosslinkable polymer polyethylene glycol diacrylate or diacrylamide with a specified molecular weight and concentration.

Beyond its foundational composition, the hydrogel can be further enriched by incorporating hyaluronic acid or aloe vera. Precise concentration ratios are crucial, with the monomer concentration falling within 10-20% w/v, the monomer to crosslinker ratio at 1:100 to 1:1000, and the photo-initiator concentration ranging from 0.01-0.1% w/v. The physical network components also require meticulous calibration, with sodium alginate at 1-5% w/v, calcium carbonate at 7.5-60 mM, and D-glucono-δ-lactone at 15-120 mM, wherein the viscosity of sodium alginate at 1% should be between 50-500 mPa·s.

The crosslinking process is executed under UV light, with flexibility in choosing from UVA, UVB, or UVC light. Specifically, UVC light with a wavelength of 200-280 nm is highlighted as a preferred option. This method achieves a hydrogel with versatile applications, combining form-fitting attributes, biocompatibility, and tunable mechanical properties, setting it apart in the realm of hydrogel technology.

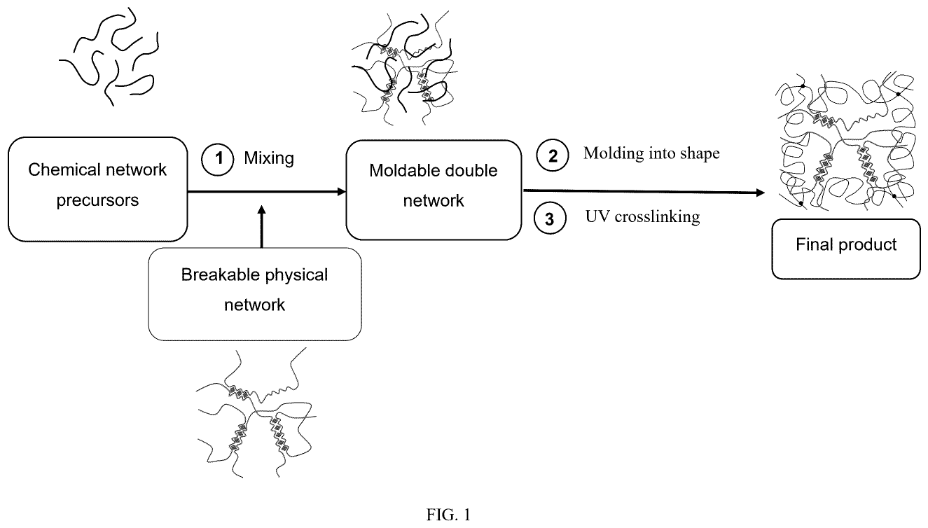

Referring to FIG. 1, chemical network precursors are formed, which are prepared by mixing a monomer, a crosslinker and a photo-initiator or a crosslink-able polymer and a photo-initiator. A force-sensitive “breakable” physical network is obtained by mixing sodium alginate, calcium carbonate and D-glucono-δ-lactone. The chemical network precursors are mixed with the breakable physical network together to form a moldable double network. At this stage, the moldable double network can be molded into any shape for any specific applications. Once the double network is poured into a mold and is ready for gelation, a UV light will be provided to start crosslinking between the two networks to form the final product, a double network system hydrogel with tunable mechanical properties.

As used herein, the term “breakable” refers to the bonding or the crosslinking between the molecules or compounds is disruptable, particularly the physical network, rather than describing a physical break into two of a hydrogel.

It is important to note that the addition of calcium carbonate and D-glucono-δ-lactone is to control the timing of Ca²⁺ availability to achieve homogenous mixing before gelation.

Further, the selected photo-initiator is water-soluble and highly efficient, so that the crosslinking time is shortened from 1-3 hours to less than 15 minutes. In one embodiment of the present invention, the photo-initiator is a water-soluble photo-initiator, such as lithium phenyl-2,4,6-trimethylbenzoylphosphinate and 1-[4-(2-Hydroxyethoxy)-phenyl]-2-hydroxy-2-methyl-1-propane-1-one.

In accordance with a second aspect of the present invention, a form-fitting and biocompatible hydrogel is prepared by the above method.

This form-fitting and biocompatible hydrogel, with a pH range of 4.0-8.5 at room temperature, is designed to ensure non-cytotoxicity, rendering it completely safe and gentle on the skin.

The hydrogel exhibits a finely tuned mechanical profile, encompassing an elastic modulus ranging from 10-50 kPa, an elongation at break spanning 100-300%, and a residual strain falling within the 0-35% range. This combination of mechanical characteristics ensures the hydrogel's compatibility with the dynamic and flexible nature of biological tissues.

Moreover, the hydrogel showcases versatility by seamlessly incorporating hyaluronic acid and/or aloe vera, enhancing its utility and potential benefits. The hydrogel can effectively absorb and retain up to 2 wt % of these substances, allowing for personalized formulations based on specific requirements or desired functionalities.

Additionally, the crosslinked chemical network forming the hydrogel comprises polymers of varying molecular weights. This diversity in molecular structures contributes to the hydrogel's overall stability, resilience, and tailored performance, making it suitable for a wide array of applications across medicine, skincare, and beyond.

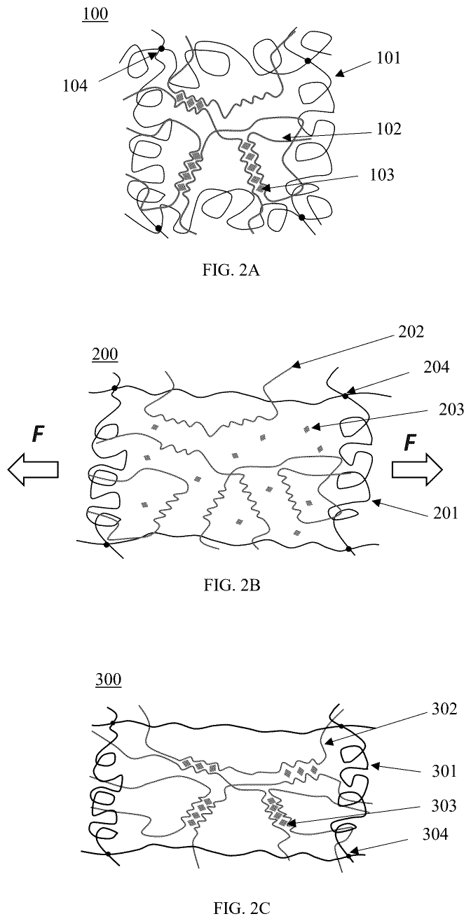

Referring to FIGS. 2A-2C, the structures of a hydrogel of one embodiment at different states are shown. As shown in FIG. 2A, the tunable hydrogel 100 is at a normal state. The chemical network includes monomers or polymers 101, crosslinkers 104 and photo-initiator (not shown) and the physical network includes sodium alginate 102, calcium ions 103 from calcium carbonate and D-glucono-δ-lactone (not shown). The chemical network is built upon chemical covalent bonds, and the physical network is formed due primarily to the ionic interaction between calcium and alginate. Two networks are mixed and interengaged evenly and topologically such that force can transmit from one network to the other. Referring to FIG. 2B, the tunable hydrogel 200 is enduring forced extension. When the hydrogel 200 is pulled by two ends, the physical network is stretched and broken, but the chemical network remains intact. It shows that the physical network of this double network hydrogel plays an important role for absorbing external forces and then dissipating energy by partially breaking to maintain the structure of hydrogel, so that the hydrogel shows a high stretchability, up to 300% strain. Further referring to FIG. 2C, it shows the form-fitting property of the hydrogel 300. After the hydrogel 300 is stretched for applying to a surface, such as the human skin in a face mask or wound dressing, due to the protection of the physical network, the hydrogel is able to be stretched without breaking; following stretching, the hydrogel will conform to its applied surface as it attempts to return to its unstretched state. In one embodiment of the present invention, upon elongation to 300% or less, the hydrogel is able to recover to its original shape.

The tunability of the hydrogel is due to the selection of the amount of physical networks in relation to the amount of chemical networks. To create a hydrogel that is more elastic, stretchable, and size-adjustable, more physical networks are included in the hydrogel, and the chemical network should have a lower crosslink density. For example, this can be achieved by increasing the alginate concentration, the acrylamide to bisacrylamide ratio, or the molecular weight of the crosslinkable polyethylene glycol. To create a hydrogel that is less elastic, stretchable, and size-adjustable, fewer physical networks are included in the hydrogel, and the chemical network should have a higher crosslink density. This can be achieved by decreasing the alginate concentration, the acrylamide to bisacrylamide ratio, or the molecular weight of the crosslinkable polyethylene glycol.

In one embodiment, the crosslinked chemical network includes polymers of different molecular weights.

In accordance with a third aspect of the present invention, a wound dressing including the above-mentioned hydrogel is provided. Particularly, the wound dressing might be optionally in a sterile form

In accordance with a fourth aspect of the present invention, a facial mask including the above-mentioned hydrogel and a skincare solution is provided.

In one embodiment, the skincare solution is active ingredients in a liquid form. For example, the active ingredients include hyaluronic acid and aloe vera, and the liquid form may be lotion, essence or toner.

### EXAMPLES

### Example 1. Polyacrylamide (PAm) Based Hydrogel Preparation

PAm based hydrogel is prepared by using acrylamide as monomer and bisacrylamide as crosslinker with sodium alginate, CaCO₃, and D-glucono-δ-lactone. Samples 1-16 has different ratios of acrylamide and bisacrylamide, and the exact components and corresponding mechanical properties are listed in Table 1.

TABLE 1
PAm based hydrogel samples

| | | Ratio of | | | | | |
|---|---|---|---|---|---|---|---|
| | | acrylamide and | | | | | |
| | Acrylamide | bisacrylamide | Alginate | CaCO₃ | Modulus | Elongation | Residual |
| No. | (w/v %) | (wt %) | (wt %) | (mM) | (kPa) | (%) | strain (%) |
| 1 | 12 | 250 | 1 | 10 | 12.7 (±0.8) | 200 (±16) | 5.85 (±0.68) |
| 2 | 12 | 250 | 2 | 15 | 19.0 (±0.9) | 190 (±8) | 13.1 (±1.11) |
| 3 | 12 | 1000 | 1 | 20 | 10.5 (±0.5) | 128 (±1) | 19.6 (±0.9) |
| 4 | 14 | 1000 | 3 | 20 | 18.2 (±1.3) | 252 (±11) | 20.1 (±0.9) |
| 5 | 15 | 100 | 1 | 10 | 47.6 (±1.2) | 100 (±9) | 3.58 (±0.25) |
| 6 | 16 | 250 | 0.5 | 7.5 | 20.3 (±0.4) | 123 (±15) | 2.73 (±0.16) |
| 7 | 16 | 250 | 1 | 10 | 23.8 (±0.7) | 160 (±16) | 3.96 (±0.19) |
| 8 | 16 | 250 | 1.5 | 12.5 | 25.7 (±0.9) | 136 (±7) | 4.30 (±0.49) |
| 9 | 16 | 250 | 2 | 15 | 32.4 (±1.1) | 132 (±28) | 5.41 (±0.79) |
| 10 | 16 | 400 | 2.5 | 35 | 58.5 (±5.6) | 177 (±19) | 16.5 (±0.1) |
| 11 | 16 | 600 | 1 | 15 | 12.4 (±0.8) | 303 (±9) | 24.1 (±0.5) |
| 12 | 17 | 400 | 2.5 | 18 | 31.4 (±4.3) | 205 (±15) | 14.0 (±0.4) |
| 13 | 17 | 600 | 1 | 10 | 17.3 (±1.4) | 243 (±2) | 12.8 (±0.3) |
| 14 | 17 | 600 | 2.5 | 35 | 48.7 (±2.2) | 207 (±17) | 24.9 (±0.3) |
| 15 | 17 | 800 | 2.5 | 35 | 45.2 (±1.6) | 292 (±26) | 30.9 |
| 16 | 18 | 600 | 2.5 | 18 | 26.6 (±1.0) | 281 (±12) | 19.1 (±0.4) |

It is found that an increase in alginate concentration increases the elastic modulus of the hydrogel, and the elongation at break does not change significantly with the increasing alginate concentration, showing that the elongation at break remains similar at low alginate content (0-2 wt %). Further, the higher alginate concentration can result in a higher residual strain.

Furthermore, the increase in acrylamide content leads to increase in the elastic modulus, and the decrease in bisacrylamide content leads to a decrease in the elastic modulus. Elongation at break is also increased while the amount of bisacrylamide is decreased.

### Example 2. Polyethylene Glycol (PEG) Based Hydrogel

The hydrogel provided by the present invention can be prepared by using PEG as well. The detailed components of PEG based hydrogel and the tested results of mechanical property thereof are listed in Table 2.

TABLE 2

| | PEGDA- | PEGDA- | PEGDA- | Alginate | CaCO₃ | Modulus | Elongation | Residual |
|---|---|---|---|---|---|---|---|---|
| No. | 8k (%) | 10k (%) | 20k (%) | (%) | (mM) | (kPa) | (%) | strain (%) |
| 1 | 4 | — | 8 | 2 | 15 | 39.9 (±1.9) | 256 (±8) | 9.0 |
| 2 | 4 | — | 10 | 2 | 15 | 45.2 (±2.3) | 259 (±8) | 19 |
| 3 | 4.5 | — | 9 | 2 | 15 | 49.0 (±3.0) | 174 (±22) | 4.5 (±0.7) |
| 4 | 7.5 | — | 2.5 | 2 | 15 | 39.2 (±2.7) | 132 (±19) | 2.3 (±1.5) |
| 5 | — | 6 | — | 2 | 15 | 24.5 (±1.4) | 184 (±15) | 7.0 (±2.6) |
| 6 | — | 6 | — | 2 | 30 | 31.6 (±3.6) | 213 (±21) | 22.1 (±2.6) |
| 7 | — | 7 | — | 2 | 15 | 25.7 (±1.2) | 195 (±39) | 13.7 (±1.2) |
| 8 | — | 7.5 | 2.5 | 2 | 15 | 39.2 (±2.6) | 179 (±34) | 0.7 (±1.2) |
| 9 | — | 8.5 | — | 1 | 7.5 | 20.7 (±0.2) | 207 (±55) | 1.5 (±2.1) |
| 10 | — | 8.5 | — | 1 | 10 | 25.3 (±0.6) | 161 (±56) | 2.0 |
| 11 | — | 8.5 | — | 2 | 15 | 31.6 (±1.8) | 159 (±14) | 5.0 |
| 12 | — | 10 | — | 2 | 15 | 37.4 (±3.9) | 170 (±31) | 6.7 (±0.6) |
| 13 | 2.5 | — | 5 | 2 | 15 | 20.3 (±4.4) | 264 (±60) | 17.5 (±0.7) |
| 14 | — | 3.5 | 7 | 2 | 15 | 23.7 (±1.5) | 251 (±45) | 15.3 (±4.5) |
| 15 | — | 4 | 8 | 2 | 15 | 32.2 (±5.1) | 278 (±25) | 12.3 (±1.2) |

An increase in the polymer content leads to an increase in the gel elastic modulus. With the increase in molecular weight of PEG, elongation at break is increased. The mechanical properties can be controlled by using a mixture of PEGs with different molecular weights. For example, an increased fraction of PEGDA-8K in the mixture leads to a lower elongation at break; the greater the level of PEGDA-20K, the higher the elongation at break and residual strain. Therefore, the mixtures of PEGDA-20K, 10K and 8K allow to fine tune the elongation and residual strain within the lower- and upper-bounds set by the single PEG systems.

### Example 3. The Incorporation of Skin Calming Additives

It is further evaluated whether the mechanical properties of Pam based hydrogel are affected by incorporating skin treatment materials, for example, skin calming additives that reduce skin inflammation into the hydrogel. Elastic modulus, elongation at break and residual strain of the hydrogel sample No. 12 of Pam based hydrogel prepared by acrylamide, bisacrylamide, alginate and CaCO₃ are first evaluated. After adding 2% hyaluronic acid or aloe vera, the same mechanical properties are tested again. The results are shown in Table 3.

TABLE 3
Mechanical properties of Pam based hydrogel with additives.

| | Elastic modulus | Elongation at | Residual |
|---|---|---|---|
| Sample No. 12 | (kPa) | break (%) | strain (%) |
| Control | 31.4 ± 4.3 | 205 ± 15 | 14.0 ± 0.4 |
| +2% hyaluronic | 33.0 ± 2.5 | 205 ± 5 | 13.0 ± 1.9 |
| acid | | | |
| change | 5.10% | 0% | −7.14% |
| +2% aloe vera | 32.3 ± 4.5 | 211 ± 5 | 15.1 ± 0.9 |
| change | 2.87% | 2.93% | 7.86% |

The results show that there is less than a 10% change in the elastic modulus, elongation at break and residual strain of the hydrogels after incorporating hyaluronic acid (2 wt %) and aloe vera (2 wt %).

Furthermore, the mechanical properties of PEG based with the incorporation of skin calming additives are also investigated. The hydrogel sample No. 6 of PEG based hydrogel prepared by PEGDA-10k, alginate and CaCO₃ is selected for evaluating elastic modulus, elongation at break and residual strain. After adding 2% hyaluronic acid or aloe vera, the same mechanical properties are tested again. The results are shown in Table 4.

TABLE 4
Mechanical properties of PEG based hydrogel with additives.

| | Elastic modulus | Elongation at | Residual |
|---|---|---|---|
| Sample No. 6 | (kPa) | break (%) | strain (%) |
| Control | 31.6 ± 3.6 | 213 ± 21 | 20.5 ± 3.0 |
| +2% hyaluronic | 33.1 ± 3.5 | 215 ± 16 | 19.5 ± 1.3 |
| acid | | | |
| change | 4.72% | 0.94% | −4.64% |
| Control | 33.5 ± 4.5 | 182 ± 16 | 16.5 ± 0.4 |
| +2% aloe vera | 34.8 ± 2.4 | 200 ± 0 | 17.0 ± 0.4 |
| change | 3.88% | 9.89% | 3.02% |

As same as Pam based hydrogel, the results show that there is less than a 10% change in the elastic modulus, elongation at break and residual strain of the hydrogels after incorporating hyaluronic acid (2 wt %) and aloe vera (2 wt %).

In summary, the hydrogel of the present invention is capable of incorporating skin calming additives without substantially affecting the mechanical properties.

### Example 4. Biocompatibility of the Hydrogel

The hydrogel provided by the present invention has passed the industrial standard test ISO 10993-5 and ISO 10993-10 to be proven as non-cytotoxic and not harmful to the skin. The pH value of the hydrogel is also monitored, showing that the hydrogel has a pH value in the range of 4.0-8.5 at room temperature.

### Example 5. Shelf-Life of the Hydrogel

To evaluate the shelf life of hydrogel, an accelerated aging test is conducted. In the test, the accelerated aging condition is equivalent to 1 year at room temperature. The results show that the hydrogel maintains similar mechanical properties after 1 year aging (Table 4).

TABLE 4
Accelerated aging test

| | Elastic modulus | Elongation at | Residual |
|---|---|---|---|
| | (kPa) | break (%) | strain (%) |
| Before aging | 37.2 ± 2.9 | 273 ± 35 | 14.1 ± 0.8 |
| After aging | 39.1 ± 2.1 | 281 ± 19 | 15.1 ± 1.9 |
| Change | 4.99% | 2.69% | 7.09% |

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A method of manufacturing a form-fitting and biocompatible hydrogel with tunable mechanical properties, wherein the hydrogel comprises a chemical network and a physical network, comprising:
mixing a monomer, a crosslinker and a photo-initiator or a crosslink-able polymer and a photo-initiator to obtain a chemical network precursor;
mixing sodium alginate, calcium carbonate and D-glucono-δ-lactone to form a physical network precursor having ionic crosslinking;
introducing the physical network precursor into the chemical network precursor and mixing them evenly to form a moldable double network; and
providing UV light to crosslink the chemical network precursor to form a crosslinked polymer chemical network interengaged with the ionic crosslinked physical network to obtain a form-fitting and biocompatible hydrogel.

2. The method of claim 1, wherein calcium carbonate and D-glucono-δ-lactone are used for controlling the timing of Ca²⁺ availability to achieve homogenous mixing before gelation.

3. The method of claim 1, wherein by adding the photo-initiator, crosslinking time is shortened to less than 10 minutes.

4. The method of claim 1, wherein the tunable mechanical properties comprise elastic modulus, elongation at break and residual strain.

5. The method of claim 1, wherein the monomer is acrylamide.

6. The method of claim 1, wherein the crosslinker is N, N′-methylenebisacrylamide.

7. The method of claim 1, wherein the crosslink-able polymer is polyethylene glycol diacrylate or diacrylamide with a molecular weight of 8-20 kDa and a concentration of 5-15% w/v.

8. The method of claim 1, wherein the hydrogel is capable of incorporating hyaluronic acid or aloe vera.

9. The method of claim 1, wherein the concentration of the monomer is 10-20% w/v and the monomer to crosslinker ratio is 1:100 to 1:1000, and the concentration of the photo-initiator is 0.01-0.1% w/v.

10. The method of claim 1, wherein the concentration of sodium alginate is 1-5% w/v, the concentration of calcium carbonate is 7.5-60 mM and the molar concentration of D-glucono-6-lactone is 15-120 mM, wherein the sodium alginate has a viscosity of 50-500 mPa·s at 1%.

11. The method of claim 1, wherein the UV light is selected from UVA, UVB or UVC light.

12. The method of claim 11, wherein the UV light is a UVC light with a wavelength of 200-280 nm.

13. A form-fitting and biocompatible hydrogel with tunable mechanical properties prepared by the method of claim 1, wherein the hydrogel has a pH value in a range of 4.0-8.5 at room temperature, and the hydrogel is non-cytotoxic and compatible to skin.

14. The hydrogel of claim 13, wherein the hydrogel has an elastic modulus in range of 10-50 kPa, an elongation at break in range of 100-300% and a residual strain in range of 0-35%.

15. The hydrogel of claim 13, wherein the hydrogel is capable of incorporating with hyaluronic acid and/or aloe vera for up to 2 wt %.

16. The hydrogel of claim 13, wherein the crosslinked chemical network includes polymers of different molecular weights.

17. A wound dressing comprising the hydrogel of claim 13.

18. The wound dressing of claim 17, wherein the wound dressing is in sterile form.

19. A facial mask comprising the hydrogel of claim 13 and a skincare solution.

20. The facial mask of claim 19, wherein the skincare solution comprises hyaluronic acid and aloe vera.
